SEQUENCE LISTING (6 sequences)

SEQ ID NO: 1 - DNA, 21 bp, Artificial Sequence
  GAPDH siRNA sequence
ugguuuacauguuccaauatt

SEQ ID NO: 2 - DNA, 21 bp, Artificial Sequence
  GAPDH siRNA sequence scrambled sense
ugguuuacaugucgacuaatt

SEQ ID NO: 3 - DNA, 20 bp, Artificial Sequence
  GAPDH FW primer
tgccctcaacgaccactttg

SEQ ID NO: 4 - DNA, 20 bp, Artificial Sequence
  GAPDH REV primer
ccaccaccctgttgctgtag

SEQ ID NO: 5 - DNA, 21 bp, Artificial Sequence
  HTRP1 FW primer
gcgatgtcaataggactccag

SEQ ID NO: 6 - DNA, 22 bp, Artificial Sequence
  HTRP1 REV primer
ttgttgtaggatatgcccttga

(19) United States
(12) Patent Application Publication
(10) Pub. No.: US 2024/0245720 A1
(43) Pub. Date: Jul. 25, 2024

(54) COMPOSITIONS COMPRISING EXTRACELLULAR VESICLES AND AN ACTIVE AGENT AND USES THEREOF

(71) Applicant: RAMOT AT TEL-AVIV UNIVERSITY LTD., Tel Aviv (IL)

(72) Inventors: Daniel OFFEN, Tel Aviv (IL); Nataly YOM-TOV, Tel Aviv (IL)

(21) Appl. No.: 18/564,356

(22) PCT Filed: May 25, 2022
(86) PCT No.: PCT/IL2022/050553

§ 371 Date: Nov. 27, 2023

Related U.S. Application Data
(60) Provisional application No. 63/192,672, filed on May 25, 2021.

(51) Int. Cl. A61K 31/785; A61K 9/50; A61K 9/51; B82Y 5/00

(52) U.S. Cl. CPC A61K 31/785; A61K 9/5068; A61K 9/5146; A61K 9/5161; B82Y 5/00

(57) ABSTRACT

The present disclosure provides a composition comprising an extracellular vesicle (EV) associated with an active agent and a polymer, wherein said polymer carries at least one positively charged moiety or has at least one positive pole and said active agent carries, independently, at least one negatively charged moiety or has at least one negative pole; and wherein said polymer and said active agent are present at a positive to negative charge or pole ratio of at least 2. A preferred example relates to a composition wherein the polymer is polyethyleneimine, the agent is a nucleic acid and the EV is an exosome. The present disclosure also provides methods for obtaining the composition and uses of the composition.

### TECHNICAL FIELD

The present disclosure relates to drug delivery.

### BACKGROUND ART

References considered to be relevant as background to the presently disclosed subject matter are listed below:
Fu et al., NanoImpact 2020, 20:100261; Jafari et al., BioDrugs 2020, 34:567-586
Pizzicannella et al. Front Physiol 2019, 10:512
Zhupanyn et al J Control Release 2020, 319:63-76
U.S. Patent Application Publication No. 2018/0154023
International Patent Application Publication No. WO20180107061
International Patent Application Publication No. WO2021222133
International Patent Application Publication No. WO2021154205
U.S. Pat. No. 10,765,638
International Patent Application Publication No. WO2016178233
International Patent Application Publication No. WO2011/013130

Acknowledgement of the above references herein is not to be inferred as meaning that these are in any way relevant to the patentability of the presently disclosed subject matter.

### BACKGROUND

Extracellular vesicles (EVs) are particles with a lipid bilayer that are naturally released from a cell, but which cannot replicate. EVs may be released from the surface of cells, in which case they are referred to as ectosomes, microvesicles or microparticles; or in endosomal compartments which release the EVs when the endosomal compartment fuses with the cell surface, in which case they are referred to as exosomes. EVs often comprise proteins and RNA (e.g., micro RNAs), and are hypothesized to play a natural role in cell-to-cell signaling.

Exosomes have been considered by Fu et al., as promising carriers for drug loading and delivery, due to their ability to cross various biological/physical barriers such as the blood-brain barrier (BBB), stability and non-immunogenicity (which protects their cargo), non-toxicity relative to synthetic nanoparticles, and ability to target specific sites. Exosomes have been loaded with nucleic acids via co-incubation of exosomes and nucleic acids; by transfection of exosome-producing cells; or by electroporation.

International Patent Application Publication WO 2018/011153 describes the use of cell penetrating peptides (CPPs) to carry agents, such as siRNA, mRNA and peptides, into EVs.

Pizzicannella et al. describes EVs coated with PEI in order to promote osmotic swelling and burst of endosomes containing the EVs, thereby promoting release of the EV content into cells.

Zhupanyn et al describes a combination of PEI/siRNA complexes of about 110-160 nm in size with EVs of about 100-200 nm in size to form EV-modified PEI/siRNA complexes of about 170-220 nm in size. According to the teachings of this document, the complexes exhibit improved knockdown efficacy compared to the non-modified PEI/siRNA complexes and storage stability for 5 days.

US2018/0154023 describes lyophilization and reconstitution of compositions comprising complexes between a nucleic acid and a cationic polymer such as PEI.

WO20180107061 describes negatively charged lipid-based nanoparticles (e.g. exosomes) having (a) a core comprising a cationic polymer and a therapeutic agent and (b) a lipid coating comprising an exosome-derived membrane. Also described are methods of producing and methods of use of such particles.

WO2021222133 describes methods and systems for producing polymer-DNA nanoparticles of a predetermined size. The method involves mixing a first solution comprising DNA with a second solution comprising a cationic polymer and adding to the polyplex formed a stabilizing agent. The resulting particles can then be transfected.

WO2021154205 describes a transfection reagent prepared by ionic interaction of colostrum powder-derived exosome and a polycation. The resulting exosome-polycation matrix is entrapped with biological material such as nucleic acid.

U.S. Pat. No. 10,765,638 describes compositions and methods for delivery of therapeutic agents in vivo. The compositions are in the form of polymeric particles comprising a therapeutic agent complexed with a polycationic polymer which is further encapsulated in one or more amphiphilic polymers.

WO2016178233 describes compositions comprising a nucleic acid, a cationic polymer, and a carbohydrate solution, wherein the nucleic acid and the cationic polymer form a complex and wherein the w/w ratio of the carbohydrate to the nucleic acid-polymer complex is between 50 and 1,000.

Finally, WO2011/013130 describes a method for generating a particle for delivery of polynucleotides to a target cell, the particles contain a covalently bound complex.

### SUMMARY OF THE INVENTION

The present disclosure provides in accordance with a first of its aspects a composition comprising an extracellular vesicle (EV) associated with an active agent and a polymer,
wherein said polymer carries at least one positively charged moiety or has at least one positive pole and said active agent carries, independently, at least one negatively charged moiety or has at least one negative pole,
and wherein said polymer and said active agent are present at a positive to negative charge or pole ratio of at least 2.

In one particular example, the present disclosure provides a composition comprising exosomes, each exosome being associated with a nucleic acid and linear PEI, wherein said PEI and said nucleic acid are present at a ratio between amine groups of said PEI and phosphate groups of said nucleic acid of at least 2.

Also provided by the present disclosure is a method of stably associating between extracellular vesicles (EVs) and an active agent, the method comprises incubating a suspension with a solution of said EVs to thereby obtain an incubation medium comprising said EV's associated with said active agent and said polymer;
wherein said suspension comprises nanoparticles comprised of said active agent complexed with a polymer;
wherein said polymer carries at least one positively charged moiety or has at least one positive pole and said active agent carries at least one negatively charged moiety or has at least one negative pole; and
wherein said polymer and said active agent are present in said nanoparticles at a positive to negative charge or pole ratio of at least 2.

In a particular example, the present disclosure provides method of stably associating between exosomes and negatively charged nucleic acid, the method comprises incubating a suspension comprising nanoparticles comprised of said negatively charged nucleic acid complexed with polyethyleneimine (PEI) with a solution of said exosomes, to thereby obtain an incubation medium comprising said exosomes associated with the nucleic acid and PEI; and
wherein said PEI and said nucleic acid are present in said nanoparticles at a positive to negative charge ratio of at least 2.

In a further aspect, the present disclosure provides a method comprising contacting a biological cell or tissue with the composition disclosed herein, under conditions facilitating introduction of at least the negatively charged active agent into the biological cells or tissue cells. The method can be any one of in vitro, ex vivo and in vitro.

In yet a further aspect, the present disclosure provides a therapeutic method comprising administering to a subject in need of treatment the composition disclosed herein in an amount sufficient to provide a beneficial therapeutic effect on said subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the disclosure may be practiced.

FIG. 1 is a Nanosight analysis of MSC-EVs concentration and size distribution, showing a total concentration of 2.06e+08+/−1.39e+07 particles/ml and particles size of less than 300 nm.

FIGS. 2A-2B are fluorescence activated cell sorting (FACS) analysis of EVs expression of surface molecules, after 50 μl of EVs were incubated with 12.5 μl of 4-μm-diameter aldehyde/sulfate latex beads and stained with CD63-APC or CD81-APC Abs (right curve) or negative control IgG1 Isotype Ab (left curve).

FIG. 3 is a transmission Electron Microscope image of a single EV according to an example of the present disclosure, showing an EV diameter of less than 200 nm.

FIG. 4 is a bar graph showing fatty acid amide hydrolase (FAAH) activity in cells contacted with exosomes loaded with siRNA that is capable of inhibiting FAAH or with scrambled siRNA according a non limiting example of the present disclosure, or with non-loaded exosomes or PEI/siRNA nanoparticles (with FAAH siRNA or scrambled siRNA) which were not used to load exosomes (as a percentage of the activity in the negative control of untreated cells).

FIG. 5 is a bar graph showing glyceraldehyde-3-phosphate dehydrogenase (GAPDH) activity in cells contacted with exosomes loaded with siRNA capable of inhibiting GAPDH (siGAPDH) at two different exosome concentrations 5×10\hat{ }8 (low) or 5×10\hat{ }9 (high) or scrambled siRNA according to another non limiting example of the present disclosure, or with non-loaded exosomes or PEI/siRNA nanoparticles (“Complex”).

FIG. 6 is a FACS analysis showing the PEI loading efficacy using fluorescently-labelled siRNA. EVs were incubated with magnetic beads conjugated to CD81 Antibodies. The FACS analysis was carried to determine FITC positive signal of the magnetic beads.

### DETAILED DESCRIPTION

The present disclosure is based on the development of extracellular vesicles (EV) effectively loaded with an active agent; whereby the loaded agent maintains its functionality even after long term storage of the loaded EVs.

Thus, according to a first of its aspects, the present disclosure provides a composition comprising an extracellular vesicle (EV) associated with an active agent and a polymer;
wherein said polymer carries at least one positively charged moiety or has at least one positive pole and said active agent carries, independently, at least one negatively charged moiety or has at least one negative pole;
and wherein said polymer and said active agent are present at a positive to negative charge or pole ratio of at least 2.

Extracellular vesicles (EVs) are particles with a lipid bilayer that are naturally released from a cell, but which cannot replicate. EVs may be released from the surface of cells, in which case they are referred to as ectosomes, microvesicles or microparticles; or in endosomal compartments which release the EVs when the endosomal compartment fuses with the cell surface, in which case they are referred to as exosomes.

EVs modulate cell-to-cell communication in normal physiology and pathology by presenting their contents (primarily RNAs, proteins, and lipids) to recipient cells in target tissues.

Besides their natural biological properties, EVs such as exosomes are used as carriers for drug loading and delivery, due to their ability to cross various biological/physical barriers such as the blood-brain barrier (BBB), stability and non-immunogenicity (which protects their cargo), non-toxicity relative to synthetic nanoparticles, and ability to target specific sites.

Thus, in the context of the present disclosure, the term EV should be understood to define any cell derived particles having an internal space surrounded by a cell lipid membrane.

As used herein, the term “cell-derived” refers to an EV produced within, by or from a biological cell. The EV may be derivable from the cell by any of several means, for example by secretion, budding or dispersal from the cell. For example, the EV may be produced, exuded, emitted or shed from the cell. Where the cell is in cell culture, the particle may be secreted into the cell culture medium.

The EVs preferably have at least one property of the cell it is derived from. The EVs may have a biological property, such as a biological activity.

The cells from which the EVs may be derived from may be of any source and or any tissue origin. Non-limiting examples of tissues include neural tissue, kidney tissue, lung tissue, bone marrow, cord blood, adipose tissue, dental pulps.

According to some of any of the examples described herein, the cell is a mammalian cell. According to specific examples, the cell is a human cell.

The cell may be a primary cell or an immortalized cell line.

According to some of any of the examples described herein, the cell is a primary cell. Non-limiting examples of cells that are usable in the context of the present embodiments include neuronal cells, kidney cells, hematopoietic cells, adipocytes.

The cell may be a fully differentiated cell or a stem or progenitor cell.

According to some of any of the examples described herein, the cell is a stem or progenitor cell.

As used herein, the term “stem or progenitor cell” refers to a cell capable of undergoing mitotic division and differentiating into other cell types having a particular, specialized function (e.g., fully differentiated cells); and includes e.g. a totipotent cell, a pluripotent cell or a multipotent cell and may refer to a cell committed to a specific lineage.

Non-limiting Examples of stem or progenitor cells from which the EVs may be derived from include, embryonic stem cells, induced pluripotent stem cells (iPS), adult stem or progenitor cells, bone marrow-derived stem or progenitor cells, hematopoietic progenitor cells, mesenchymal stem cells (MSCs), neuronal stem cells (NSCs), neural crest cell (NCC), oral mucosa stem cells.

According to some of any of the examples described herein, the stem or progenitor cell is selected from the group consisting of a mesenchymal stem cell (MSC), neuronal stem cells (NSC) and neuronal crest cell (NCC).

There are different types of EVs. Non-limiting examples of EVs that can be used according to some exemplary embodiments of the present disclosure include an exosome, ARRM, microvesicle, exomere, membrane particle, membrane vesicle and extosome.

According to some specific examples, the EV is an exosome.

According to some examples, the EV is a mesenchymal stem cell (MSC)-derived exosome.

The EV may have a size greater than 30 nm. At times, the EV has a size within a range of from 30 nm and 1,000 nm; at times, between 30 nm and 500 nm; t times, between 30 nm and 400 nm; at times, between 30 nm and 300 nm; at times, between 30 nm and 200 nm.

In some examples, the EV have a size of equal or below 200 nm.

The size may be determined by various means. In principle, the size may be determined by size exclusion methods, for example, by size fractionation and filtration through a membrane with the relevant size cut-off. The EV size may then be determined by tracking segregation of component proteins with SDS-PAGE or by a biological assay. Whenever a “size” of an EV is described herein, it refers to at least one dimension of the EV, for example, diameter, and it refers to an average size of a plurality of EVs.

The size of the EV may alternatively by reflected as its hydrodynamic radius. The hydrodynamic radius of the particle may be determined by any suitable means, for example, laser diffraction or dynamic light scattering.

In some examples, the size of the EV is determined using transmission Electron Microspore (TEM).

The EVs are associated with an active agent and a polymer.

By “associated with” it is meant that the EV, the active agent and the polymer are in association with one another. In the context of the present disclosure, the association is a non-chemical, i.e. non-covalent association. In other words, the association is a physical association including, without being limited thereto, encapsulation, entrapment, entanglement deposition, absorption, etc, an any combination of these types of association. For example, the active agent and polymer can be surface adsorbed as well as encapsulated within the vesicles internal core.

The association between the EV and the polymer/active agent is a stable association. In the context of the present disclosure, when referring to a stable association it is to be understood to encompass physical and/or chemical and/or biological (functional) stability after storage for at least 30 days, at times, 35 days, at times 40 days, at times 45 days, at times 50 days.

In some examples, the stability is at least in terms of retained or exhibited activity of the active agent after storage for at least 30 days at 4ºC.

The active agent and the polymer can be associated with the EV in various manners. In some examples, the active agent and the polymer are in a form of a complex one with the other, i.e. an agent/polymer complex. The complexation is a non-covalent association between the two entities.

In the context of the present disclosure, when referring to a complex it is to be understood to encompass any form of non-covalent aggregation/association between the polymer and the active agent.

In some examples, a plurality of complexes of the active agent and of the polymer are complexed and aggregated into a nanoparticle. For simplicity, in the description hereinabove and below, when referring to a complex it is to be understood to include also other forms of association, including aggregation into nanoparticles.

In the context of the present disclosure, the polymer is a physiologically acceptable polymer.

In some examples, the polymer comprises at least one positively charged moiety. The polymer can be a cationic polymer, i.e. having a net positive charge, or a zwitterion, i.e. a polymer that contains an equal number of positively- and negatively-charged moieties.

In some examples, the polymer is a cationic polymer. In some examples, the cationic polymer has more than one positively charged moiety (i.e. plurality of positively charged moieties).

Exemplary cationic polymers include, but are not limited to, polyethyleneimine, polyallylamine, polyetheramine, polyvinylpyridine, polysaccharides having a positively charged functionalities thereon, polyamino acids, poly-L-histidine, poly-D-lysine, poly-DL-lysine, poly-L-lysine, poly-e-CBZ-O-lysine, poly-e-CBZ-DL-lysine, poly-e-CBZ-L-lysine, poly-OL-ornithine, poly-L-ornithine, poly-DELTA-CBZ-DL-ornithine, poly-L-arginine, poly-DL-alanine-poly-L-lysine, poly(-L-histidine, L-glutamic acid)-poly-DL-alanine-poly-L-lysine, poly(L-phenylalanine, L-glutamic acid)-poly-DL-alanine-poly-L-lysine, poly(L-tyrosine, L-glutamic acid)-poly-DL-alanine-poly-L-lysine, copolymers of L-arginine with tryptophan, tyrosine, or serine, copolymers of D-glutamic acid with D-lysine, copolymers of L-glutamic acid with lysine, ornithine, or mixtures of lysine and ornithine, and poly-(L-glutamic acid).

The cationic polymers, such as polyethyleneimimine (PEI), polylysine and diethylethanolamine (DEAE)-dextran can be used to transfect cells with molecules of a counter charge, e.g. negatively charged nucleic acids. The relatively negative charged groups (at least partially negatively-charged groups) of the nucleic acid bind to the cationic polymer.

In some examples, the polymer is a cationic polymer is or comprises a polyalkylimine.

In some examples, the polyalkylimine is selected from the group consisting of linear polyethyleneimine (PEI), branched PEI, poly L-lysine (PLL).

In some examples, the polyalkylimine is PEI. PEI may be linear (having the formula (—CH₂CH₂NH—)n), wherein almost all amines are secondary amines; or branched, in which case various combinations of primary, secondary and tertiary amines may be present.

In some examples, the PEI is a linear PEI.

The size of the polymer can vary and should not be a limiting parameter. Yet, in some examples, the polymer or the PEI has an average weight of at least 5 kDa, at least 10 kDa, or at least 15 kDa. In some examples, the PEI has an average weight of up to 40 kDa, at times up to 35 kDa, at times up to 30 kDa. In some examples, the PEI has an average weight in the range of 20 kDa±10 kDa.

In some examples, the polymer is uncharged and yet has a positive pole via which it can interacted with the negative charge or negative pole of the active agent.

Non-limiting examples of polymers with a positive pole include modified (uncharged) PEI, having a positive pole as a result of the modification. The modification can include chemical linkage to another entity, such as an antibody, receptor, ligands etc., resulting in a chemical conjugate, or the modification can involve complexation with another polymer, such as such as, Poly-(Lactic-co-glycolic Acid) (PLGA) polyplex with PEI leads to the desired electrostatic positive pole.

The active agent has at least one negatively charge moiety or has a negative pole.

By “negatively charged moiety or has a negative pole” it is meant a chemical moiety or group that is capable of featuring a temporary (transient) or permanent negative charge, that is, a moiety that features two atoms that have substantially different electronegativity and features a temporary or permanent dipole moment in which the more electronegative atom has a higher electron density than the less electronegative atom. The more electronegative atom is thus capable of complexing to positively charged groups of the cationic polymer via electrostatic interactions.

In some examples, the active agent has at least one negatively charged moiety. The agent can have an overall net negative charge, or it can be a zwitterion, i.e. an agent that contains an equal number of positively- and negatively-charged moieties.

In some examples, the active agent is selected from a saccharide, a nucleic acid, nucleotide mono-di and triphosphate, nucleoside mono-, di- and triphosphate, as well as any analogue where the phosphate is substituted with an analogue entity.

In some examples, the agent is a nucleic acid (which features, for example, negatively charged, as described herein, phosphate groups).

The nucleic acid can be, for example, DNA or RNA, including siRNA, micro RNA, mRNA, guide RNA, Small activating RNAs (saRNAs) and plasmid.

The term “nucleic acid” encompasses sequences of the naturally-occurring nucleobases and also encompasses sequences that include any of the known base analogs of DNA and RNA such as 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, uracil-5-oxyacetic acid, queosine, 2-thiocytosine and 2,6-diaminopurine.

In some embodiments, the active agent is or comprises one or more nucleobase analogs such as, but not limited to, 4-acetylcytosine, 8-hydroxy-N6-methyladenosine, aziridinylcytosine, pseudo isocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydro uracil, inosine, N6-isopentenyladenine, 1-methyladenine, 1-methylpseudouracil, 1-methylguanine, 1-methylinosine, 2,2-dimethylguanine, 2-methyladenine, 2-methyl guanine, 3-methylcytosine, 5-methylcytosine, N6-methyladenine, 7-methylguanine, 5-methylaminomethyluracil, 5-methoxy-aminomethyl-2-thiouracil, 5′-methoxycarbonyl methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, uracil-5-oxyacetic acid, queosine, 2-thiocytosine and 2,6-diaminopurine.

In some examples, the nucleic acid is a siRNA.

In some examples, the nucleic acid is a micro RNA.

In some examples, the nucleic acid is a mRNA.

In some examples, the nucleic acid is a guide RNA.

In some examples, the nucleic acid is a saRNAs.

In some embodiments, the nucleic acid is a plasmid.

In some embodiments, the active agent is a therapeutic agent, i.e. that has been proven, under acceptable criteria, to provide a therapeutic benefit.

In some examples, the therapeutic agent is a nucleoside-based drug, such as, without being limited thereto, Remdesivir or Tenofovir.

The active agent and the polymer can interact via a charge-to-charge interaction, i.e. the polymer having at least one positively charged moiety and the agent having at least one negatively charged moiety; or via a charge to pole interaction, i.e. one of the polymer or agent having a charge and the other being uncharged and yet exhibiting a dipole moment, and thereby can interact with the charge of the agent or polymer, respectively.

In some examples, the polymer has a positively charged moiety or has a net positive charge and the active agent has a negatively charged moiety or has a net negative charge.

The polymer and the active agent are present in the composition disclosed herein at a positive to negative charge or pole ratio of at least 2.

By the term “positive to negative charge or pole ratio” it is to be understood to mean that the overall charge or polarity of the polymer is greater (by at least 2) that the charge or polarity of the active agent, thus providing a net positive charge or polarity to the resulting complex of the polymer and active agent.

The ratio can be determined by various ways. For example, it can be calculated based on the mass and number of monomer units of a polymer from which the charge or pole can be created and dividing the number of charges/poles of the polymer by the charges/poles of the active agent.

In some examples, the positive to negative charge or pole ratio of at least 3, at times at least 4; at times, at least 5; at times, at least 6; at times, at least 7; at times, at least 8; at times, at least 9; at times, at least 10. In some examples, the positive to negative charge or pole ratio of up to 30.

In some examples, the positive to negative charge or pole ratio of between 3 and 10; at times, between 4 and 10; at times, between 5 and 10.

In some examples, the polymer is PEI, preferably linear PEI and the active agent is a nucleic acid. In this particular non-limiting example, the PEI and the nucleic acid are present at a ratio between amine groups of the PEI and the phosphate groups of the nucleic acid of at least 2; or at least 3; or at least 4; or at least 5; or at least 6; or at least 7; or at least 8; or at least 9; or at least 10. In some examples, the ratio is not more than 30.

In some examples, the resulting association between the polymer and the active agent results in a positively charged complex or a complex having an overall positive polarity and this is associated with the EV to form the composition disclosed herein.

When the polymer and active agent are in a form of a complex, the complex can also be characterized by its dimensions. In some examples, the dimensions of the complex is at least smaller than the dimensions of the EV with which it is associated.

In some examples, the complex is in a form of nanoparticles having dimensions of less than 200 nm; at times, less than 180 nm; at times, less than 160 nm; at times, less than 140 nm; at times, less than 120 nm; at times, less than 100 nm; at times, less than 80 nm.

In some examples, the complex (nanoparticles) are at least 20 nm.

The stable association between the EV and the polymer/active agent was surprising particularly in view of the dimensions ratio between the EV and that of the complex of the polymer/active agent when forming the association. Specifically, it has been found that relatively large polymer/active agent nanoparticles were successfully associated with exosomes. The association between the polymer/active agent complexes and the EV was challenging and it has been found to depend, inter alia, on the overall positive net charge or polarity of the complex.

With respect to the successful association between the polymer/active agent complex and the EV, the present disclosure thus also provides a method for associating the extracellular vesicles (EVs) with at least the active agent as defined herein. The method comprises incubating a suspension comprising the active agent complexed with a polymer, with a solution comprising the EVs; to thereby obtain an incubation medium comprising the EV's associated with said active agent and said polymer;
wherein the polymer carries at least one positively charged moiety or has at least one positive pole as hereinabove defined with respect to the disclosed composition; and the active agent carries at least one negatively charged moiety or has at least one negative pole as hereinabove defined with respect to the disclosed composition; and
wherein the polymer and the active agent are present in the nanoparticles at a positive to negative charge or pole ratio of at least 2, as hereinabove defined with respect to the disclosed composition.

The association between the EV and the polymer/active agent is a stable association, as defined hereinabove.

It is noted that, at times, the complexation between the polymer and the active agent can be facilitated by controlling the pH and/or providing a proton rich environment. Nonetheless, the pH of the eventual composition comprising the EV with the associated active agent does not necessarily need to be the same pH used for the complexation. The pH or the desired charge/polarity can be determined based on the specific polymer and/or active agent to be complexed.

In some examples, suspension comprises nanoparticles where the active agent is complexed with the polymer.

In some examples, the suspension comprises at least one carbohydrate. In some examples, the carbohydrate within the suspension is a saccharide (including di, tri, oligo and polysaccharides) or polyvinylpyrolidone.

In some examples, the carbohydrate hash the general formula (CH₂O)ₙ and an therefore interchangeably be used with the term “saccharide”, “polysaccharide”, “oligosaccharide” and “sugar”, as is well known in the art.

The carbohydrate may be any one of mono- di-, tri- and oligo-saccharides, as well polysaccharides such as glycogen, cellulose, and starches.

Exemplary carbohydrates include, but are not limited to, monosaccharides such as glucose, fructose, mannose, xylose, arabinose, galactose, and others; disaccharides such as trehalose, sucrose, cellobiose, maltose, lactose and others; oligosaccharides such as raffinose, stacchyose, maltodextrins and others; polysaccharides such as cellulose, hemicellulose, starch and others, and any combination thereof.

In some examples, the carbohydrate is selected from trehalose, glucose, sucrose, lactose, mannitol, sorbitol, raffinose, PVP, and dextrose.

In some examples, the carbohydrate is a monosaccharide or a disaccharide.

In some examples, the carbohydrate is a saccharide such as trehalose, glucose, sucrose, lactose, mannitol, sorbitol, or raffinose, or any combination thereof.

In some examples, the at least one carbohydrate is trehalose.

The suspension of nanoparticles and the EV's are incubated for a period of time and under conditions that facilitate the stable association between the EV and the nanoparticles. Without being bound by theory, it is believed that the polymers and the active agent are at least partially retained as nanoparticles or in the form of a complex within the EV. However, it may also be the case that once within the EV, at least a portion of the polymer and the active agent are no longer complexed together.

In some examples, the conditions of incubation include incubation at room temperature (25° C.±3° C.).

After incubation period, the EVs associated with the active agent and the polymer are collected. The collection can be by any means that would not damage the EVs per se. In some examples, the collection of the EVs associated with the active agent/polymer is by filtration.

In some examples, the collection of the EVs associated with the active agent is by passing the incubation mixture through a filter membrane (with a pre-defined cutoff) to collect the loaded exosomes.

In some examples, the membrane is characterized by a cutoff in a range of from 10 kDa to 100 kDa.

In some examples, when the EV is an exosome, the membrane can be one having a 30 kDa cutoff.

In some examples, the collection of the EVs associated with the active agent also results in the separating of the free active agent/polymer nanoparticles from the EVs.

In some examples, the suspension of the nanoparticles is obtained or obtainable by a method comprising adding a first solution comprising the active agent to a second solution comprising the polymer to form the nanoparticle suspension. The nanoparticles obtained have the positive to negative charge or pole ratio of at least 2.

In some examples, the nanoparticles are obtainable by a method as described in US patent application publication No. 2018/0154023 (corresponding to WO2016/178233), which is incorporated by reference as if fully set forth herein.

In some examples, at least a portion of the nanoparticles are loaded into the inner core of the EV. In some examples, at least a portion of the active agent is loaded into the inner core of the EV.

In some particular examples, there is provided a method for the stable association between exosomes and negatively charged nucleic acid, the method comprises incubating a suspension comprising nanoparticles comprised of the negatively charged nucleic acid complexed with polyethyleneimine (PEI) with a solution of the exosomes, to thereby obtain an incubation medium comprising the exosomes associated with the nucleic acid and PEI; wherein said PEI and said nucleic acid are present in said nanoparticles at a positive to negative charge ratio of at least 2, at times, at least 3, at times, at least 4; at times, at least 5; at times, at least 6; at times, at least 6.

The composition disclosed herein can have different beneficial uses, which require the introduction of the active agent into a cell.

Thus, in accordance with some examples, the composition is for use in achieving a therapeutic effect that is facilitated by the introduction of the active agent into a cell.

In some examples, the composition is for use in treating a condition treatable by the active agent. The treatment with the composition provides an effect that is greater than the effect achievable when the active agent is administered without being associated with the EV. In some examples the beneficial (e.g. therapeutic) effect obtained by the composition disclosed herein is statistically significantly greater than the effect achieved by the complex without the EV.

In the context of the present disclosure the condition for which the composition can be used should not be limited and in fact any condition for which treatment is desired by an active agent as defined herein falls within the scope of the present disclosure.

In some examples, the condition is a viral infection, and the active agent is Remdesivir.

In some examples, the effect provided by the composition disclosed herein exhibits a synergistic action between the active agent and the EV, i.e. the beneficial effect (e.g. therapeutic effect) exhibited by the EV associated with the active agent or the nanoparticle comprising the agent and the polymer is higher than the sum of (combined) effects of the active agent or nanoparticle and the EV, when the active agent (or nanoparticle) and the EV are each administered alone.

Also provided by the present disclosure are methods.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In some aspects of the present disclosure the method is a method comprising contacting a biological cell or tissue with the composition disclosed herein, under conditions facilitating introduction of at least the active agent into the biological cells or tissue cells.

In some other aspects of the present disclosure the method is a method of treatment, the method comprises administering to a subject in need of treatment an amount of the composition disclosed herein, the amount being sufficient to provide a beneficial therapeutic effect on said subject.

In the context of the present disclosure, when referring to “administering” or “administration” to a subject it is to be understood to encompass any means of administration of EV as known in the art.

Without being limited thereto, the composition can be administered intranasally, subcutaneously (SC), intravenously (IV), intraperitoneally (IP), by inhalation, in the form of eye drops etc.

In some examples, the administration is by inhalation.

In some examples, the administration is by intravenous injection or infusion.

In some examples, the administration is subcutaneously (SC).

In some examples, the administration is by topical administration as eye drops.

In some examples, the administration is intranasally.

All definitions, as defined and used herein, should be understood to control over dictionary definitions, definitions in documents incorporated by reference, and/or ordinary meanings of the defined terms.

The term “about” if and when used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to +10%.

The indefinite articles “a” and “an” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

Throughout this description, Examples and claims which follow, all transitional phrases such as “comprising” “including” “carrying” “having” “containing” “involving” “holding” “composed of” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various examples of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging ranges between” a first indicate number and a second indicate number and “ranging ranges from” a first indicate number “to” a second indicate number are used herein interchangeably and are meant to include the first and second indicated numbers and all the fractional and integral numerals there between.

It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub combination or as suitable in any other described embodiment of the present disclosure. Certain features described in the context of various embodiments are not to be considered essential features of those embodiments, unless the embodiment is inoperative without those elements.

Various embodiments and aspects of the present disclosure as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present disclosure will be limited only by the appended claims and equivalents thereof.

The following examples are representative of techniques employed by the inventors in carrying out aspects of the present disclosure. It should be appreciated that while these techniques are exemplary of preferred embodiments for the practice of the invention, those of skill in the art, in light of the present disclosure, will recognize that numerous modifications can be made without departing from the spirit and intended scope of the present disclosure.

### NON-LIMITING EXAMPLES

Reference is now made to the following examples, which together with the above descriptions illustrate some embodiments of the invention in a non-limiting fashion.

### Exosomes Preparation

Exosomes derived from adipose-derived mesenchymal stem cells (AdMSCs), obtained from human fat tissue donation cultured in an exosomes-depleted Fetal Bovine Serum (FBS).

The isolation and purification of the resulting AdMSC-Exo from the conditioned medium was by continued centrifuges protocol.

In brief, the conditioned medium was centrifuge for 10 minutes at 300 g, and the supernatant was re-centrifuged for another 10 minutes at 2,000 g, followed by re-centrifuged at 10,000 g for 30 minutes. The recovered supernatant was passed through 0.22 μm filters to isolate the exosomes from debris and dead cells' residue. The exosomes were then purified from the filtrate by ultracentrifuge at 100,000 g for 2 h. The pellet containing the exosomes was resuspended in 6000 μl of 5% trehalose and then passed through 30 kDa Amicon—to discharge the remaining proteins outside the exosomes.

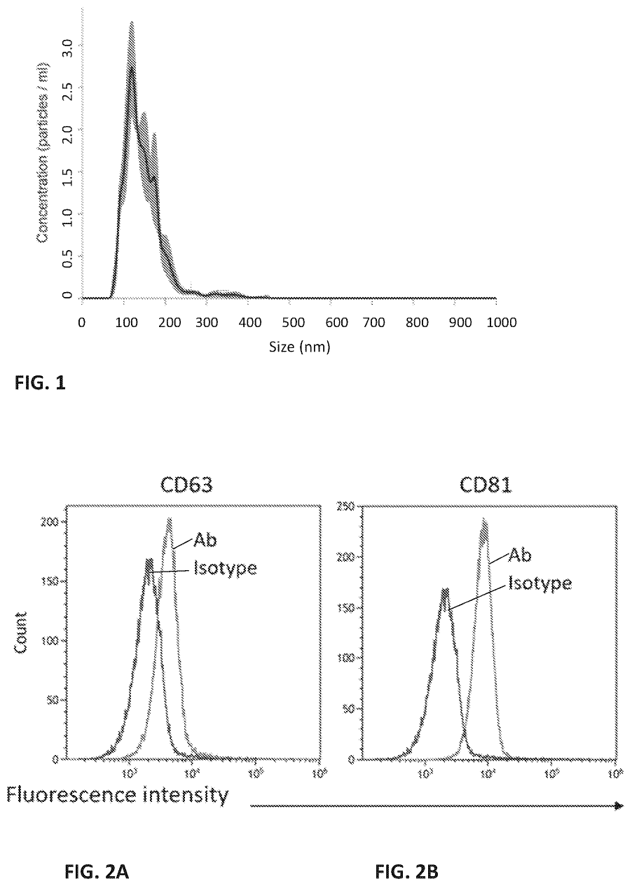

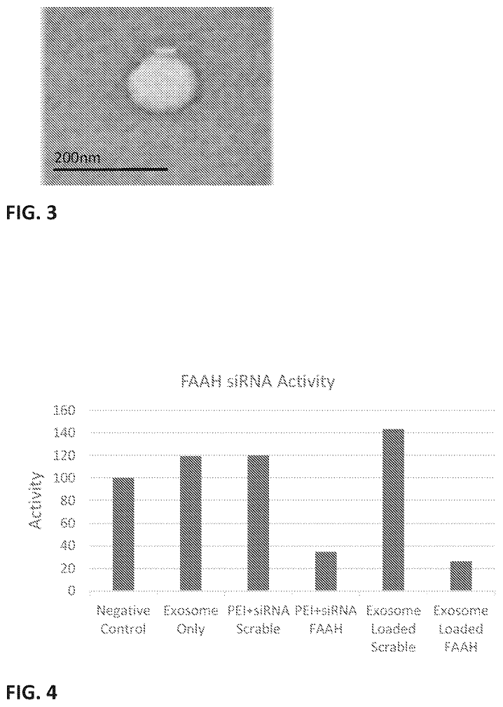

Conventional methods determined the purity and integrity of the purified exosomes. Specifically,
Nanosight technology (performed according to manufacturer instructions) was used to measure the concentration and the mean size of the exosomes. See FIG. 1 1 showing a total concentration of 2.06e+08+/−1.39e+07 particles/ml and a size below 200 nm. It is noted that the total concentration was determined after dilution and therefore the actual concentration was 2.06e+09+/−1.39e+08 particles/ml.
Fluorescence-activated cell sorting (FACS) analysis used to confirm the expression of exosomal marker CD63 and CD81 using latex beads conjugated to CD63 and CD81 antibodies, as shown in FIGS. 2A and 2B, respectively.
Transmission electron microscopy (TEM) was used to visualize the exosomes directly, as shown in FIG. 3, with an EV size of less than 200 nm (according to indicated scale)
   Preparation of PEI and siRNA Nanoparticles (Complexation)

Complexation of PEI and siRNA was performed in a low-polarity environment (5% trehalose aqueous solution), as described in U.S. Patent Application Publication No. 2018/0154023 the content of which is incorporated herein by reference.

Briefly, 0.06 ml of PEI (in vivo-jetPEI®, comprising linear PEI, obtained from Polyplus), were added to 0.565 ml Trehalose 5% and thoroughly mixed. siRNA solution (0.125 ml of 4 μg/ul siRNA in 0.5 ml of Trehalose 5%) was added dropwise into the PEI solution to form PEI-siRNA nanoparticles sufficiently small for loading exosomes.

### Loading of PEI-siRNA Nanoparticles in Exosomes

Isolated exosomes from human adipocytes mesenchymal stem cells (0.100 ml contains 2.5×10\hat{ }11 exosomes in 2 ml Trehalose 5%) were incubated for 1 hour (at room temperature) with 0.250 ml PEI-siRNA nanoparticles in order to load the exosomes.

### FAAH Activity Inhibition

The siRNA used was fatty acid amide hydrolase (FAAH) inhibiting siRNA (“FAAH siRNA”). As control, scrambled siRNA was used.

The PEI-siRNA complex was prepared as described above.

N2A cells (CRL-1972 from ATCC) were contacted with the loaded exosomes. As controls, N2A cells were directly contacted with PEI-siRNA nanoparticles, as well as non-loaded exosomes and scrambled siRNA.

In order to assess the degree of inhibition by the siRNA, the FAAH activity in the N2A cells was determined as follows:

Real-time reverse transcription quantitative PCR (RT-qPCR) was performed in order to determine mRNA levels in the exosomes loaded with the PEI-siRNA nanoparticles, and in the control groups of PEI-siRNA nanoparticles (without exosome) non-loaded exosomes and scramble RNA.

RT-qPCR was performed in order to determine mRNA levels between the treatment groups comparing to no treatment sample. (mRNA was extracted using RNeasy Kit Qiagen, followed by 1 ug mRNA to cDNA using Bio-Rad iScript kit. cDNA was then compared using RT-qPCR).

As shown in FIG. 4, exosomes loaded with FAAH siRNA were at least as effective as nanoparticles comprising PEI and FAAH siRNA at in vitro reducing cellular FAAH activity, reducing FAAH activity by about 75%, as compared to about 65% by nanoparticles. It is noted that the non-loaded exosomes and scrambled siRNA controls had no inhibitory effect).

### Storage Stability Assay

Storage stability (i.e. activity after storage) was evaluated by periodically testing the activity of loaded exosomes as described herein. A stock of nanoparticles of PEI with siRNA-GAPDH was prepared as described above and stored it in 4ºC.

The PEI-siRNA-GAPDH was then associated with the exosomes. To this end, two solutions of 1.9 ml were prepared containing Trehalose 5% and 0.1 ml exosomes containing 10\hat{ }11 exosomes or 0.01 ml exosomes containing 10\hat{ }10 exosomes. To the exosome solution 0.25 ml PEI-siRNA (GAPDH) nanoparticles were added. The resulting mixture was mixed for 1 h at 4° C. in rotation, and stored under 4° C.

For testing GADPH inhibition capacity, several samples were taken from the stored stock, in several time point up to 61 days. Specifically, the activity was determined by adding 0.1 ml of the mixture (PEI siRNA-GAPDH and exosomes at two different concentrations) to 293HEK cells in a 6 wells plates_B, (300,000 cells for well). The cells were incubated for 48 hours.

The GAPDH levels were assayed by RT-PCR. (GAPDH siRNA sequence as denoted by SEQ ID NO 1: UGGUUUACAUGUUCCAAUAdTdT for the scrambled sense as denoted by SEQ ID NO 2: UGGUUUACAUGUCGACUAAdtdt Dharmacon, USA)

Then we proceeded to RT-qPCR analysis. Generally, the assay included collecting the cells, washing with PBS, adding trypsin and centrifuging the cells in 1100 RPM for 5 min. The cells pellet was subject for RNA extraction according to the kit protocol. Nanodrop was used to determine RNA concentration and to this 1 ug of RNA was used for cDNA preparation using iScript and adding 180 Rnase Dnase free water to each cDNA tube to reach 200 ul total. For the Real-Time reaction the following primers were used:
GAPDH FW as denoted by SEQ ID NO 3: TGCCCTCAACGACCACTTTG
GAPDH REV as denoted by SEQ ID NO 4: CCACCACCCTGTTGCTGTAG
HTRP1 FW as denoted by SEQ ID NO 5: GCGATGTCAATAGGACTCCAG
HTRP1 REV as denoted by SEQ ID NO 6: TTGTTGTAGGATATGCCCTTGA
qPCR reaction was done according to manufactures SYBER protocol.

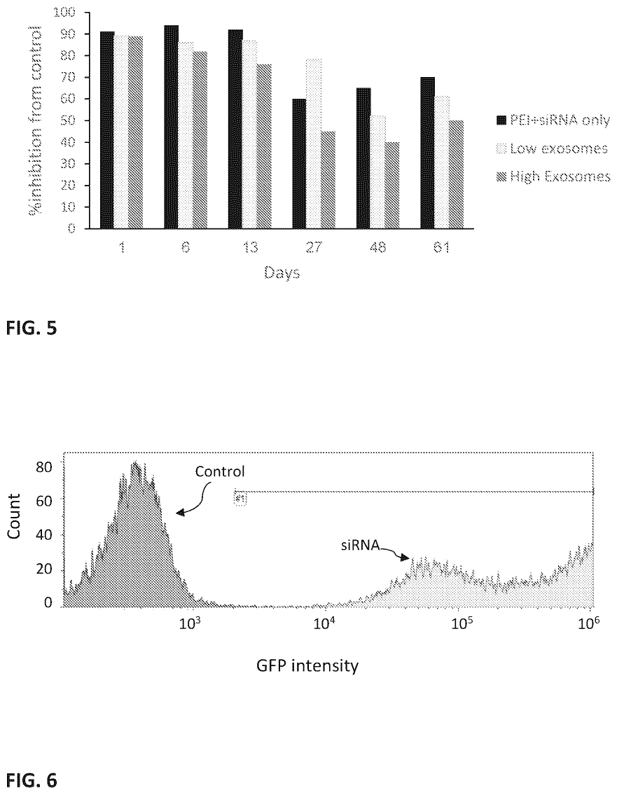

FIG. 5 shows % inhibition from control at the different time points. For each time point nanoparticles alone, nanoparticles with exosomes 5×10\hat{ }8 (low exosome concentration) or 5×10\hat{ }9 (high exosome concentration) were tested. In this connection, it is noted that when using “low” exosome concentration, the loading of the PEI-siRNA is in fact high. The scrambled RNA was tested only after day one and it was found that the inhibition was only 9% (data not shown).

FIG. 5 shows that EVs loaded with GAPDH siRNA reduced the GAPDH expression levels ranging from 90% in the first time points to 50% after 61 days.

Further, FIG. 5 shows that for when using the mixture that originally contained a lower concentration of exosome (and thus a higher nanoparticles/exosome ratio), the % inhibition was higher, i.e. higher loading and thus higher inhibition of expression (higher activity of the resulting loaded exosome).

Finally, FACS analysis was conducted to show the PEI loading efficacy using fluorescently-labelled siRNA (FIG. 6). EVs were incubated with magnetic beads conjugated to CD81 Antibodies. The FACS analysis was carried to determine FITC positive signal of the magnetic beads. About 90% positive events were recorded across wide range of signal intensity.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-55. (canceled)

56. A composition comprising an extracellular vesicle (EV) associated with an active agent and a polymer,
wherein said polymer carries at least one positively charged moiety or has at least one positive pole and said active agent carries, independently, at least one negatively charged moiety or has at least one negative pole;
and wherein said polymer and said active agent are present at a positive to negative charge or pole ratio of at least 2.

57. The composition of claim 56, wherein said polymer comprises one of the following (i) at least one positively charged moiety; (ii) a net positive charge; (iii) at least one positive pole.

58. The composition of claim 56, wherein said active agent comprises one of the following: (i) at least one negatively charged moiety (ii) at least one negative pole.

59. The composition of claim 56, wherein said polymer and said active agent comprise, respectively, a positive or negative charge or at least one of said polymer and said active agent is uncharged.

60. The composition of claim 56, wherein said active agent and said polymer are complexed together in a form of nanoparticles; said particles having a size of not more than 100 nm.

61. The composition of claim 56, wherein said active agent is a biopolymer.

62. The composition of claim 56, wherein said polymer is a polyalkylimine.

63. The composition of claim 56, wherein at least part of said active agent and said polymer are encapsulated within said EV.

64. The composition of claim 56, wherein said EV is an exosome.

65. A composition comprising exosomes, each exosome being associated with a nucleic acid and linear PEI, wherein said PEI and said nucleic acid are present at a ratio between amine groups of said PEI and phosphate groups of said nucleic acid of at least 2.

66. A method of associating between extracellular vesicles (EVs) and an active agent, the method comprising incubating a suspension comprising nanoparticles comprised of said active agent complexed with a polymer, with a solution of said EVs to thereby obtain an incubation medium comprising said EV's associated with said active agent and said polymer;
wherein said polymer carries at least one positively charged moiety or has at least one positive pole and said active agent carries at least one negatively charged moiety or has at least one negative pole; and
wherein said polymer and said active agent are present in said nanoparticles at a positive to negative charge or pole ratio of at least 2.

67. The method of claim 66, wherein said suspension comprises a carbohydrate.

68. The method of claim 67, wherein said nanoparticles are obtained or obtainable by a method comprising adding a first solution of the active agent to a second solution of the polymer, wherein said second solution comprises a carbohydrate which may be the same or different from a carbohydrate in said suspension.

69. The method of claim 66, wherein said active agent is a biopolymer.

70. The method of claim 67, wherein said polymer is a polyalkylimine.

71. The method of claim 66, wherein said EVs are exosomes.

72. The method of claim 66, wherein said polymer comprises one of (i) at least one positively charged moiety; (ii) a net positive charge; and (iii) at least one positive pole.

73. The method of claim 66, wherein said active agent comprises one of (i) at least one negatively charged moiety; and (ii) at least one negative pole.

74. The method of claim 66, wherein said polymer and said active agent comprise, respectively, a positive or negative charge or at least one of said polymer and said active agent is uncharged.

75. A method of stably associating between exosomes and negatively charged nucleic acid, the method comprising incubating a suspension comprising nanoparticles comprised of said negatively charged nucleic acid complexed with polyethyleneimine (PEI) with a solution of said exosomes, to thereby obtain an incubation medium comprising said exosomes associated with the nucleic acid and PEI;
wherein said PEI and said nucleic acid are present in said nanoparticles at a positive to negative charge ratio of at least 2.

76. A therapeutic method comprising administering to a subject in need of treatment a composition according to claim 56, the amount of the composition being sufficient to provide a beneficial therapeutic effect on said subject.

77. A therapeutic method comprising administering to a subject in need of treatment a composition according to claim 65, the amount of the composition being sufficient to provide a beneficial therapeutic effect on said subject.
